(19) United States
(12) Patent Application Publication
(10) Pub. No.: US 2020/0345825 A1
(43) Pub. Date: Nov. 5, 2020

(54) FORMULATION OF FISH VACCINE BASED ON LIPIDIC NANOVESICLES, IN PARTICULAR, A PROTEOLIPOSOME OR COCHLEATE, WITH ACTIVITY AGAINST THE SALMONID RICKETTSIAL SYNDROME (SRS)

(71) Applicants: UNIVERSIDAD DE CHILE, Santiago (CL); EMPRESAS NGEN LABORATORIO S.A., Santiago (CL)

(72) Inventors: Leonardo SAENZ ITURRIAGA, Santiago (CL); Mario CARUFFO MADRID, Santiago (CL); Sonia VIDAL VILCHES, Santiago (CL); Leonardo SANTIS MEZA, Santiago (CL)

(21) Appl. No.: 16/957,859

(22) PCT Filed: Dec. 20, 2018
(86) PCT No.: PCT/CL2018/000041

§ 371 Date: Jun. 25, 2020

(30) Foreign Application Priority Data
Dec. 26, 2017 (CL) 3358-2017

(51) Int. Cl. A61K 39/02; A61K 9/127

(52) U.S. Cl. CPC A61K 39/0208; A61K 9/1271; A61K 2039/55555; A61K 9/1274; A61K 9/1277

(57) ABSTRACT

The invention relates to aquaculture. In particular, it relates to immunisation in fish farming. More particularly, the present invention relates to a vaccine formulation for fish, based on lipid nanovesicles with activity. Even more particularly, the present invention relates to a vaccine formulation for fish, based on lipid nanovesicles, especially a proteoliposome, with activity against salmon rickettsial syndrome (SRS)

### FIELD OF THE INVENTION

This invention refers to aquaculture. In particular, this invention refers to immunisation in fish farming. More particularly, this invention refers to a formulation of fish vaccine based on lipidic nanovesicles with activity. In a more particular way, this invention refers to a formulation of fish vaccine based on lipidic nanovesicles, specially, a proteoliposome, with activity against the Salmonid Rickettsial Syndrome (SRS).

### BACKGROUND

Injectable vaccines for fish, based on microorganisms inactivated in oil adjuvants, began to develop in Norway at the beginning of the nineties, when vaccination by immersion against Aeromonas salmonicada was not effective. The efficacy of these vaccines produced an immediate and permanent reduction in the use of antibiotics, and these vaccination strategies remained practically unchanged for more than 10 years.

In this context the most often used vaccines in this type of production are killed vaccines (bacterins or virines) using oil adjuvants. Even though this type of vaccines presents a high safety, since they do not have the capacity to replicate in the individual, during the inactivation process the antigens are seriously damaged by the heat effect or chemical substances, decreasing substantially the capacity of the fish to set an adequate and durable acquired immunity that assure the protection of the individual during production. Also, it has been proven that they are only capable to produce a slight humoral response mediated by neutralising antibodies. By the other hand, new technologies have created vaccines formulated on the base of fragments or subunit antigens, which are extremely safe, since they do not administer whole microorganisms with the capacity to generate the disease, however, the antigens are in superior structural conditions, which substantially improves the adaptive immune response in the individuals. Despite this advantage, the delivery of specific antigens to the immune system must be sufficiently complex to stimulate the adaptive response of cellular and humoral type, to protect the individual from different pathologies.

Teleost fishes have a developed immune system similar to the mammals with certain own particularities like the centres of melanomacrophages and the phagocytic ability of the enterocytes to cite some of them, they have a first line of defence corresponding to the innate immune system as well as an adaptive immune system characterized by the presence of antigen receptors from the superfamily of immunoglobulins in the surface of lymphocytes B, macrophages and neutrophils with the antigen-presenting capacity and also response to molecular patterns associated to pathogens (PAMPs). They present adaptive immunity both cellular and humoral, being able to produce two immunoglobulins IgM and IgT, the latter is an analogue of IgA from mammals, which exerts its role at mucosal level. Humoral response is of tremendous importance in some diseases of salmonids where the development of neutralising antibodies has been described, like the case of infectious salmon anaemia (ISA) and infectious pancreatic necrosis (IPN), however, for a disease caused by a intracellular bacteria such as Piscirickettsia salmonis the formation of antibodies is not enough and it is necessary the generation of specific cellular immunity.

P. salmonisis a Gram-negative bacterium, facultatively intracellular, pleomorphic predominantly coccoid, which produces a systemic infection affecting several organs like kidney, liver, spleen, intestine, brain, ovaries and gills. Since the emergence of the disease in 1989 (https://www.ncbi.nlm.nih.gov/pubmed/9204294), this has evolved over time with more insidious outbreaks and refractory to treatments with antibiotics, which have not been able to effectively control the disease, this has meant annual losses for the national industry estimated in 100 Million of USD (http://onlinelibrary.wiley.com/doi/10.1111/jfd.12211/abstract). The intracellular location of the organism can enable that a considerable number of bacteria is out of reach of bactericidal concentrations of antibiotics, and the disease remains during the therapy. Due to limitations associated with the therapeutics, the development of effective and safe vaccines should be an element of great help in the prevention and control of the disease, however, the different formulations of vaccines against the disease available in the market present poor results of immunity and protection.

The pathologies caused by intracellular bacteria have been widely studied in pathogens like Salmonella, Listeria, Francisella and Mycobacterium and the immune responses associated with the single production of antibodies, are not capable to avoid the spread of the disease, so it is necessary to have a cellular immune response.

In order to achieve a good cellular response against an intracellular pathogen it is necessary the participation of the antigen presenting system mediated by Histocompatibility molecules (MHC I and II). The presentation in MHC II is possible thanks to the phagocytic activity of macrophages of dead bacteria or opsonized with antibodies, however, the presentation in MHC I, which is essentially cytotoxic and able to destroy cells infected with the intracellular pathogen, is widely favoured using a specialized vector for the antigen. For example, the use of nanovesicles in a vaccine against hepatitis C has been disclosed, which generates a strong response of cytotoxic lymphocytes (https://www.ncbi.nlm.nih.gov/pmc/articles/PMC4212474/#bibr44-2051013614541440). The role of liposomes and its effect in cellular responses has also been reported (https://www.ncbi.nlm.nih.gov/pmc/articles/PMC4212474/).

Among the patent documents it is possible to mention CA2888754 that refers to immunogenic compositions for mucosa and its parenteral application, which include (1) a single capsular saccharide or multiple non conjugated bacteria and (2) potent adjuvants. Oligosaccharides or single polysaccharides are preferred, but not excluding the addition of adjuvants of conjugated polysaccharides. Preferably, the adjuvants are Finlay Cochleate x (AFCox) adjuvant or Finlay Proteoliposome x (AFPLx) adjuvant. The release via mucosa is preferred, without excluding the parenteral administration or combinations of both. Particularly, it refers to immunogenic compositions that include: (a) an antigen of capsular saccharide of the serogroup C or serogroup A of Neisseria meningitidis; (b) AFPL1 adjuvants absorbed in alumina by parenteral route or not absorbed or AFPL1 AFCol by nasal route; and (c) the application of both parenteral and mucosal. The composition increases the immune response of systemic mucosa, polarising the Thymus independent response of the polysaccharides to a Thymus dependent response with a cellular pattern Thi that ensure the function of it in small children and induce immunological memory without covalent conjugation.

US2015086620 refers to a method of preparation of proteoliposomes that contact a liposome with an effective portion of RalBP1 to create a proteoliposome. RalBP1 is effective in the protection and treatment of mammals and the environment against the accumulation of toxic compounds and preventing the accumulation of them.

US2014294929 refers to a composition and method to administer charge molecules to a patient or subject who needs it, which includes as vehicle a proteoliposome with the protein RLIP76 and contains the charge molecule. The vehicle effectively releases the charge molecule systemically through the body tissues, including the central nervous system.

US2010248314 refers to a formulation of vaccine that contains proteoliposomes derived from the outer membrane of Neisseria meningitidis serogroup B (AFPLI) or the derived from cochleate (AFCol) of the same, single or in conjunction with one or more microbial or tumour antigens, and also the use of this formulation as vaccine in the prevention and treatment of infections or tumour diseases when it is administered simultaneously both for mucosa and parenteral routes of administration.

RU2009105117 refers to an immunogenic composition with activity in the serogroup B and C of Neisseria meningitidis which contains: (a) an oligosaccharide of N. meningitidis serogroup C (NMC), (b) proteoliposome vesicles of the outer membrane of N. meningitidis (NmB) of serogroup B and (c) NmB protein and a sequence of amino acids or an immunogenic fragment of this, or a sequence with 80% of identity with the previous sequence. The ingredient (a) could be conjugated with a vehicle, for example, with a protein, CRM, a diphtheritic anatoxin or a tetanus anatoxin.

JP2009292851 refers to a combined immunologic composition and a vaccine of Neisseria meningitidis B and C, and a method to trigger an immune response through its administration. The vaccine of Neisseria meningitidis includes proteins of outer membrane from the serogroup B and oligosaccharides from the serogroup C, and it is useful for the prevention or treatment of the disease. In summary, the immunogenic composition or vaccine includes NMC oligosaccharides conjugated with carrier proteins, proteins of NmB outer membrane, and a vehicle. In particular, the carrier protein is CRM197 or non-toxic diphtheria toxin, the proteins of NMB outer membrane is a proteoliposome vesicle, and the adjuvant is aluminium hydroxide or MF59. The composition could be used in the preparation of the combined vaccine that produce an immune response in both serogroups.

US2001012517 refers to proteoliposome membrane structures (MPs) useful in the preparation of specific vaccines for each patient with white blood cells (WBC) malignant tumours. The MPs typically contain a component derived from the membrane of a specific WBC. Other useful components include immunostimulators and exogenous lipids. The resulting vaccines are specific both for the patient and for the malignant tumour.

U.S. Pat. No. 4,873,089 refers to a procedure for the preparation of proteoliposomes where a fusogen is mixed with lipidic components that form unilamelar lipidic vesicles. The fusogenic unilamelar lipidic vesicles formed, are activated in the presence of integral proteins of membrane forming fusogenic proteoliposomes. These are activated by reducing the temperature of the mixture which consists in fusogenic unilamelar lipidic vesicles and integral proteins of membrane. Also, a medicine that includes the proteoliposome-fusogen mixture with one or more additional active agents is described as useful as a carrier for the drug or active principle. The proteoliposomes are bigger than conventional proteoliposomes and more stable than the conventional unilamelar lipidic vesicles.

EP1716866 refers to compositions of vaccine for the treatment or effective prevention of fungi, virus, protozoa, or bacterial infections (preferable intracellular) and cancer. It provides adjuvants for its use in prophylactic or therapeutic vaccines using bacterial proteoliposomes and their derivatives, that, when are applied with antigens which are inserted, conjugated or mixed with it, induce the response of cytotoxic T lymphocyte to the antigen. The composition could be used to obtain multiple formulations of proteoliposome or the derivatives of this with heterologous antigens that, when are applied by parenteral o mucosa route (preferable nasal), induce cytotoxic responses. it could be used to produce vaccines. In this reference, the proteoliposome is used as adjuvant.

US2016279222 refers to vesicles of the outer membrane of Francisella and Piscirickettsia, and its use in compositions of vaccine. In particular, it refers to compositions and methods useful in the induction of protective immunity against francisellosis or salmonid rickettsial septicaemia (SRS) of fish. Also, it reveals a method to provide immunity by administering to the fish a composition including a vesicle of outer membrane from a selected microorganism of Francisella spp. and Piscirickettsia spp. and a composition that includes a purified preparation of vesicles of outer membranes from Francisella spp. or Piscirickettsia spp, where this fish is selected from the group consisting of cod, Gadus morhua; tilapia, Oreochromis sp., Atlantic salmon, Salmo salar; hybrid striped bass, Morone chrysops×M. saxatilis and three-lined grunt Parapristipoma trilinineatum. The vesicles of outer membrane have been isolated, that is to say, they are in a state other than natural; or they are biologically pure, that is to say, substantially free of toxic components. The vesicles could be administered alone or in a pharmaceutical acceptable carrier. The vesicles of outer membrane could be isolated from an inoculated culture, in stationary growth phase and obtained from the supernatant harvested. The composition of the vaccine is tested in zebra fish infected with Francisella and P. salmonis. This document uses OMV obtained by a natural procedure, this is, bacteria are induced to produce the vesicles. Thus, the composition obtained is different from those proposed in this invention.

WO2016082050 refers to a cell free liquid culture medium that allows the growth of the bacteria Piscirickettsia salmonis in a minimum period of time, by the nutritional contribution of the minimum components defined with concentrations adjusted to the demand for it, which permit a maximum concentration of biomass with a high performance to be obtained. The bacterial phenotype obtained in the cell free medium preserve the levels of virulence observed in the traditional culture systems. Thus, this technology supports the development of a new procedure, faster and more economical than current systems, the generation of a product of equal or better quality, in order to obtain a biomass required for both formulations of oral and injectable vaccines.

CA2656032 refers to procedures for the culture of bacteria from the gender Piscirickettsia, selection/identification of such bacteria, as well as the fabrication of vaccines. It also refers to compositions of vaccines, formulations and bacteria that are identified and/or cultured according to this process.

Then, several vaccines that use proteoliposomes to improve the immunogenic activity of the vaccine whether in humans or animals are known. None of them refers to a vaccine against SRS. Vaccines for bacterial infections are known, including intracellular infections, against Francisella and Piscirickettsia, which use vesicles of outer membranes (US2016279222) but these are obtained naturally from the bacteria.

### BRIEF DESCRIPTION OF THE INVENTION

This invention discloses a formulation of vaccine based on lipidic nanovesicles, specially a proteoliposome, with immunopotentiator activity against the Salmonid Rickettsial Syndrome (SRS). This formulation is an alternative to traditional injectable vaccines for fish based on microorganisms inactivated in oil adjuvants, the administration by immersion or vaccines formulated on the basis of fragments or subunits antigens.

The need of this invention arises because, although inactivated vaccines present a high safety, they have lost their capacity to provide adequate and durable immunity, since during the production, they are seriously damaged by the heat factor or chemical substances. Meanwhile, vaccines formulated based on fragments or subunit antigens, despite they are extremely safe since they do not administer whole microorganisms that could generate the disease, and they are in a superior structural conditions, that it to say, not damaged, are able to improve immune response, but their delivery is complex to achieve the stimulation of cellular and humoral response and to confer protection for different pathologies.

Humoral response is of great importance in fish, particularly in some diseases affecting salmonids such as ISAV and IPN. That response is not enough for the disease caused by the intracellular pathogen Piscirickettsia salmonis, where is also needed to generate specific cellular immunity. To achieve that immunity, it is necessary to establish a vehicle for the antigens in a specialised vector. In order to establish the vehicle, this invention propose lipidic nanovesicles, that is to say, proteoliposomes. In particular, it is proposed the use of adjuvants selected from the group: Montanide 760 VG, Montanide 763 AVG, Montanide ISA711, Drakeol 6VR.

### BRIEF DESCRIPTION OF THE FIGURES

FIGS. 1A and 1B Shows transmission electron microscopy (TEM) of proteoliposomes plus membranes (A) and cochleates along with proteoliposomes and membranes (B), formulated from Piscirickettsia salmonis.

FIG. 2 Shows the IgM levels in vaccinated and non-vaccinated animals. The averages and standard deviations obtained in each sampling point of the experiment are shown by means of the indirect ELISA technique, both for the vaccinated and for the control group (Placebo). The “*” represents the sample with significant difference.

FIG. 3 Shows the results of gene expression, obtained by real-time PCR using the comparative method ΔΔCt. Gene expression of genes TRB-I, MHC-I, CD8-a, TNF-α, MHC-II and INF-γ was determined as representative genes to measure cellular response. The expression was standardised using the ELF-I gene as control gene. The graph represents the averages and standard deviation obtained from 5 animals analysed from each group.

FIGS. 4A-4C. Show the safety of the vaccines at the end of the evaluation period (300 UTA after the second dose). In this case Vaccine 1 corresponds to the one composed of proteoliposomes, membrane and cochleates in an adjuvant of water in polymer emulsion (Montanide ISA 760 VG), and the Vaccine 2 corresponds to the same antigens in an adjuvant of water in oil emulsion (Montanide ISA 763 AVG). FIG. 4A: Comparison of fish weight between groups (without significant differences), FIG. 4B: Speiberg index of the formulations, all the adherences in the vaccinated groups were under grade 2, FIG. 4C: visceral melanosis index for vaccinated groups was 1, no parietal melanosis was recorded in any group. (figures should be provided with Spanish text).

FIGS. 5A and 5B. Evaluation of vaccines efficacy. Vaccine 1 (adjuvant Seppic Montanide ISA 760 VG) and vaccine 2 (Adjuvant Seppic Montanide ISA 763 AVG) are able to reduce the percent of mortality, but vaccine 1 is more effective in the protection (Wilcoxon, ** P<0.0001).

FIG. 6 Shows an estimation of the relative percent survival at the moment when the control group reaches 60% of mortality (RPS60), for fish challenged by intraperitoneal injection. There are 2 formulations of vaccine using an aqueous adjuvant (vaccine 1 or I as defined in the FIG. 4) and an oil adjuvant (vaccine 2 or II as defined in the FIG. 4). Specifically, the control group reaches 62.8% mortality, vaccine 1 reaches 20.8% mortality and vaccine 2 reaches 35% mortality. The RPS60 of vaccine 1 is 66.9 and for vaccine 2 is 44.3.

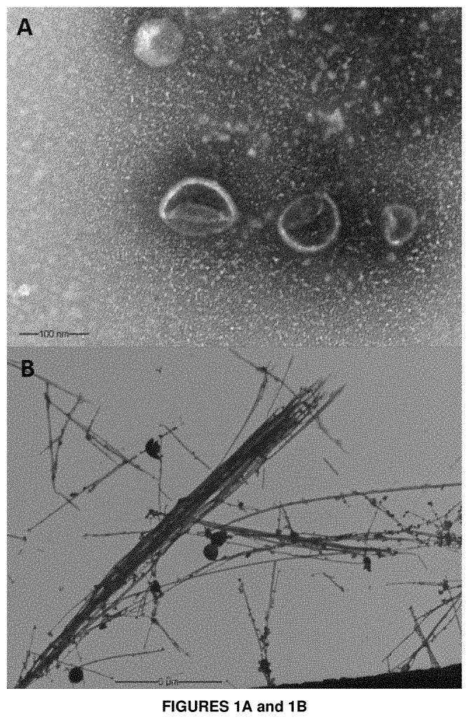

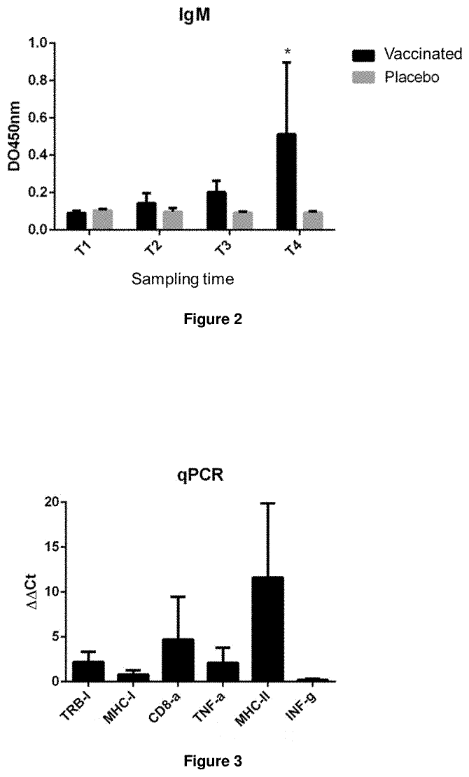

### DETAILED DESCRIPTION OF THE INVENTION

This invention discloses a formulation of vaccine based on lipidic nanovesicles, specially, proteoliposomes, with immunopotentiator activity against SRS. The composition could be for intraperitoneal injection. The formulation of vaccine generates specific cellular immunity in fish.

In particular, the measurements include the levels of antibodies (IgM) and the gene expression of TRB-I, MHC-I, CD8-α, TNF-α, MHC-II and INF-γ by ELISA, the safety of the vaccine and its efficacy and survival percent after the challenge.

The vaccine includes liposomes, bacterial membranes and fragments of cell wall of P. salmonis which have been purified from a culture previously prepared from this bacteria that then is centrifuged to obtain a sediment which is subsequently subjected to stages of freezing and thawing by sonication, in repeated steps.

In the purification stage, the frozen sediment of the microorganism is thawed and resuspended in a buffer solution adding also pearls of Zirconia/Silica 0.1 mm preferable BioSpec® to then be subject to repeated freezing and thawing by sonication until reach 7 thawings by sonication, to be later centrifuged, the sediment is discarded and the supernatant is preserved to be again centrifuged, preferable vigorous centrifugation, discarding this time the supernatant and preserving the sediment.

In the procedure of this invention, first the membrane liposomes are prepared by resuspending the sediment of the bacterial culture at pH 7.4, in a sterile solution for its solubilisation, where the sterile solution includes preferable Tris-HCl, KCl and Sodium Deoxycholate. Then, the resulting solution is incubated overnight under agitation and then it is centrifugated. The supernatant is recovered, and non-polar resins capable of capturing the detergent are added, preferable Bio-Beads®. Previously, the resins have been resuspended in a solution of Tris-HCl and KCl at pH 7.4 and sterilised by autoclave. The supernatant with the non-polar resins are incubated under agitation, the non-polar resins are decanted, and the supernatant is extracted and transferred to a sterile tube.

Regarding the membrane, this is prepared by resuspending the sediment of the bacterial culture in saline solution/physiological saline, then it is incubated with agitation and centrifugated to preserve the supernatant.

The formulation of vaccine is prepared by mixing the membrane liposomes and the membranes so that the quantity of antigen for the required dose considers 10 μg of total protein present in the membrane liposomes and 10 μg of total protein present in the membrane plus sterile saline to complete 30 μL per dose. Then, the antigen diluted in physiological saline is added to the adjuvant, gently agitating under sterile conditions. The mixture is homogenized and then stored in sealed containers to be refrigerated until its use.

The following are specific examples of preparation and testing, without limiting the scope of the invention.

The vaccine tested in rainbow trout (Oncorhynchus mykiss) challenged with P. salmonis and healthy non-immunised, showed to have a good relative percent survival at the end of the study (RPS) and a good relative percent survival when the control group reaches 60% of mortality (RPS60).

### Example 1: Preparation of Proteoliposomes, Bacterial Membranes and Cochleates

Growth of P. salmonis:

The strain of P. salmonis grew in flasks with agitation in a commercial liquid medium Grace's (Gibco), L-15 (Hyclone) or SFX-insect (Hyclone trademark).

The growth was made for 14 days, with a temperature of 18° C. and constant agitation. After the growing time, the bacteria were harvested by centrifugation, storing the sediment at −80° C.

Purification of Membranes and fragments of cell wall:

The purification of the membranes was made using the following stages:
a) The frozen sediment of the microorganism was thawed.
b) It was resuspended in 25 mL of sterile lysis buffer solution (Disodium Phosphate, Sodium Chloride; sterilised by filtration), 2 grams of pearls of Zirconia/Silica 0.1 mm (BioSpec®) were added for each gram of sediment obtained.
c) Then it was frozen at −80° C.
d) It was thawed by sonication at 400 W with Hielscher Ultrasonic Processor UP400S sonicator.
c) Then, it was frozen again.
f) The steps from d) and e) were repeated, to complete several repetitions, preferable 7 steps of sonication.
g) It was centrifugated at 200 g for 5 minutes, reserving the supernatant and discarding the sediment.
h) The supernatant was centrifuged again, but now at maximum power reserving the sediment (pellet of membranes) and discarding the supernatant.

Preparation of Membranes Proteoliposomes:

The preparation was performed following these steps:
a) The sediment was resuspended in solubilisation of membranes solution (Tris-HCl, KCl and Sodium Deoxycholate at pH 7.4 previously filtered).
b) It was incubated with agitation of 100 rpm at 18-25° C. overnight.
c) It was centrifuged at 500 g for 5 minutes and the supernatant was transferred to a new container.
d) For each 10 mL of supernatant obtained, 1 mL of non-polar resins capable to capture the detergent were added (specifically Bio-Beads® (BioRad, catalogue number 152-3920)), previously resuspended in solution Tris-HCl and KCl pH 7.4; and sterilised by autoclave.
e) It was incubated for 90 minutes at 18-25° C. with agitation of 100 rpm.
f) Sterile Bio-Beads® were added again in a proportion of 5 mL effective of Bio-Beads per each 10 mL obtained in the point c).
g) It was incubated for 90 minutes at 18-25° C. with agitation of 100 rpm.
h) Bio-Beads® were left for decantation and the supernatant was extracted with syringe and transferred to a sterile tube.

Preparation of the Membrane:

The preparation was performed following these steps:
a) The sediment was resuspended in physiological saline solution in proportion 10 mL of physiological saline per each gram of membrane.
b) It was incubated at 25° C. with agitation of 200 rpm for 72 hours.
c) It was centrifugated at 500 g for 5 minutes and the supernatant was transferred to a new container.

Preparation of Membrane Cochleates:
a) The membrane sediment was solubilised with TLis in proportion of 2 mL of TLis per each gram of sediment.
b) These solubilised membranes were incubated at 18° C. with agitation of 150 rpm for a total of 20 hours.
c) The solubilisation was centrifuged at 500 g for 5 minutes and 20° C.
d) The volume of the supernatant was measured by transferring it to a new tube.
e) The solubilised membrane was added by a constant dripping with a flow of 0.5 mL/min to a sterile beaker with 1 volume of formation solution with constant agitation with a magneto.
f) After the dripping, the agitation continued for 30 extra minutes.
g) The cochleates solution was transferred to a sterile bottle and 4 volumes of drip washing solution were added.
h) After the dripping, the agitation continued for 30 extra minutes.
i) The complete solution was centrifugated at 2000 g for 30 minutes at 20° C.
j) The supernatant was discarded, and the sediment resuspended in 0.5 volumes of washing solution.

Preparation and Bottling of the Vaccine:

The preparation was performed following these steps:
a) The quantity of antigen for the required doses was calculated considering 20 μg of total protein of equal parts of membrane, proteoliposomes and cochleates per each dose, 30-40 μL were completed with sterile saline.
b) The necessary quantity of adjuvant was placed in a beaker with bane rod considering 60-70 μL per dose (100 μL total) and agitated at 1000 rpm for 10 seconds, under sterile conditions.
c) The quantity of antigen needed diluted in sterile physiological saline from the point a) was added to the adjuvant, without stopping the agitation.
d) It was agitated slowly at 1000 rpm for 10 seconds and then at maximum power of 2121 g for 15 minutes making sure that all the mixture is homogenized.
e) Then it was filled in flasks of polypropylene of high density.
f) The flasks were sealed with rubber stoppers and stored in refrigeration until the release from quality control.

### Example 2

Growth of Piscirickettsia salmonis Strain LF89

The strain of P. salmonis grew in flasks with agitation in a commercial liquid medium Grace's (Gibco), L-15 (Hyclone) or SFX-insect (Hyclone trademark).

The growth was made for 14 days, with a temperature of 18° C. and constant agitation. After the growing period, the bacteria were harvested by centrifugation, storing the sediment at −80° C.

Bacterial Harvest

Once the growing period was completed, bacteria were harvested by centrifugation at 3.500 g during 20 minutes at 4° C. The bacterial sediment obtained is stored at −80° C. until its use.

Purification of Membranes and Fragments of Cell Wall:

The purification of the membranes was made using the following steps:
i) The frozen sediment of the microorganism was thawed.
j) It was resuspended in 25 mL of sterile lysis buffer solution (Disodium Phosphate, Sodium Chloride; sterilised by filtration), 2 grams of pearls of Zirconia/Silica 0.1 mm (BioSpec®) were added for each gram of sediment obtained.
k) Then it was frozen at −80° C.
l) It was thawed by sonication at 400 W with Hielscher Ultrasonic Processor UP400S sonicator.
m) Then it was frozen again.
n) The steps from d) and e) were repeated, to complete several repetitions, preferable 7 steps of sonication.
o) It was centrfugated at 200 g for 5 minutes, reserving the supernatant and discarding the sediment.
p) The supernatant was centrifuged again, but now at maximum power reserving the sediment (pellet of membranes) and discarding the supernatant.

Preparation of the Membrane Proteoliposomes:

The preparation was performed following these steps:
i) The sediment was resuspended in solubilisation of membranes solution (Tris-HCl, KCl and Sodium Deoxycholate at pH 7.4 previously filtered).
j) It was incubated with agitation of 100 rpm at 18-25° C. overnight.
k) It was centrifuged at 500 g for 5 minutes and the supernatant was transferred to a new container.
l) For each 10 mL of supernatant obtained, 1 mL of non-polar resins capable to capture the detergent were added (specifically Bio-Beads® (BioRad, catalogue number 152-3920)), previously resuspended in solution Tris-HCl and KCl pH 7.4; and sterilised by autoclave.
m) It was incubated for 90 minutes at 18-25° C. with agitation of 100 rpm.
n) Sterile Bio-Beads® were added again in a proportion of 5 mL effective of Bio-Beads per each 10 mL obtained in the point c).
o) It was incubated for 90 minutes at 18-25° C. with agitation of 100 rpm.
p) Bio-Beads® were left for decantation and the supernatant was extracted with syringe and transferred to a sterile tube.

Preparation of the Membrane:

For the preparation of membrane, the following these steps were performed:
k) The sediment was resuspended in physiological saline solution in proportion 10 mL of physiological saline per each gram of membrane.
l) It was incubated at 25° C. with agitation of 200 rpm for 72 horas.
m) It was centrifugated at 500 g for 5 minutes and the supernatant was transferred to a new container.

Preparation of Membrane Cochleates:
n) The membrane sediment was solubilised with TUs in proportion of 2 mL of TLis per each gram of sediment.
o) These solubilised membranes were incubated at 18° C. with agitation of 150 rpm for a total of 20 hours.
p) The solubilisation was centrifuged at 500 g for 5 minutes and 20° C.
q) The volume of the supernatant was measured by transferring it to a new tube.
r) The solubilised membrane was added by a constant dripping with a flow of 0.5 mL/min to a sterile beaker with 1 volume of formation solution with constant agitation with a magneto.
s) After the dripping, the agitation continued for 30 extra minutes.
t) The cochleates solution was transferred to a sterile bottle and 4 volumes of drip washing solution were added.
u) After the dripping, the agitation continued for 30 extra minutes.
v) The complete solution was centrifugated at 2000 g for 30 minutes at 20° C.
w) The supernatant was discarded, and the sediment resuspended in 0.5 volumes of washing solution.

Preparation and Bottling of the Vaccine:

For the preparation of membrane, the following these steps were performed:
g) The quantity of antigen for the required doses was calculated considering 20 μg of total protein of equal parts of membrane, proteoliposomes and cochleates per each dose, 30-40 μL were completed with sterile saline.
h) The necessary quantity of adjuvant was placed in a beaker with bane rod considering 60-70 μL per dose (100 μL total) and agitated at 1000 rpm for 10 seconds, under sterile conditions.
i) The quantity of antigen needed diluted in sterile physiological saline from the point a) was added to the adjuvant, without stopping the agitation.
j) This is agitated slowly at 1000 rpm for 10 seconds and then at maximum power of 2121 g for 15 minutes making sure that all the mixture is homogenized.
k) Then was filled in flasks of polypropylene of high density.
l) The flasks were sealed with rubber stoppers and stored in refrigeration until the release from quality control.

### Example 3: Experimental Design and Samples

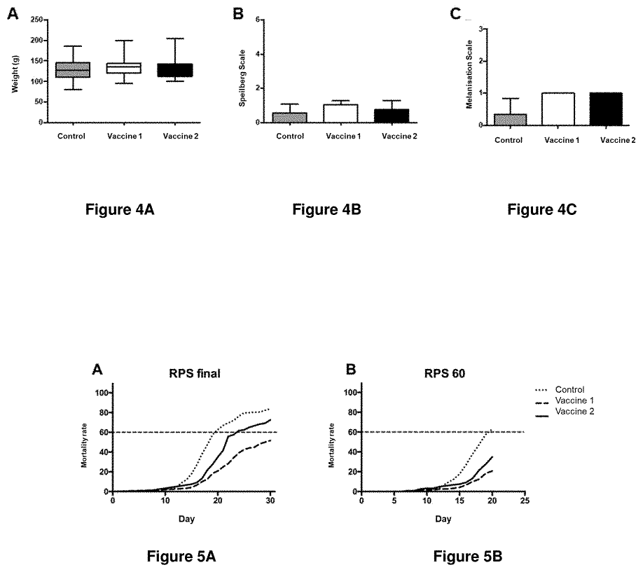

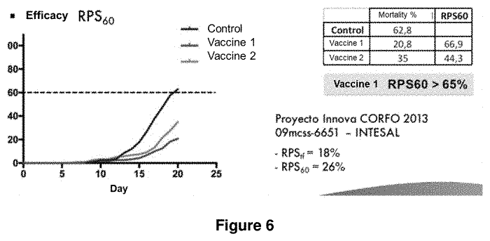

Rainbow trout (Oncorhynchus mykiss) clinically healthy and with an average weight of 40 g were maintained in a tank, with a recirculation system of fresh water and acclimatised in a controlled environment (Temperature 10 to 12° C., oxygen saturation 100-105%) during 2 weeks in 3 tanks with 400 fish each one (1000 L tanks with a density of 9 kg/m³). After the acclimatisation period, fish of every tank were vaccinated intraperitonially (0.1 mL): Tank 1: vaccine 1 as defined for FIG. 4, tank 2: vaccine 2 as defined for FIG. 4 and tank 3: control (saline solution), 300 thermal units (UTA) after the vaccination fish received a second dose.

300 UTA after the second dose 120 fish (40 per group) were transferred to common tanks of 720 L with a density of 27 kg/m³. This experimental design was also performed in triplicate.

All groups were challenged by intraperitoneal injection with 0.1 mL of Piscirickettsia salmonis (10⁴ TCID₅₀/fish). The mortality was recorded daily until day 30 post challenge and confirmed by real time PCR assays. Serum, kidneys, and spleen were sampled from 3 fish of each group at different times (T1 pre-vaccination, T2 post-vaccination, T3 post-revaccination, T4 post-challenge) for the evaluation of antibodies increase and genes expression related with cellular response.

Evaluation of Immunological Response

Antibody Response to the Vaccine

Plates of 96 wells (Nunc Maxisorp, Roskilde, Denmark) were activated with 2 μg of P. salmonis. The plates were blocked with 1% BSA and then incubated with the serums in a dilution 1:50 at 4° C. Later, they were incubated with the monoclonal secondary antibody of mice anti-salmon (dilution 1:500) IgM isotype IgG1 (BiosChile, IGSA, Chile) for 1 hour. Serums of fish experimentally infected with P. salmonis and from healthy individuals non-immunised were used as positive and negative control, respectively.

b.—Cellular Response to the Vaccine

RNA was extracted from samples of anterior kidney and spleen, using Trizol (Thermo Fisher Scientific). For the reverse transcription, 1 μg de RNA was used, according to the manufacturer's instructions (ImProm-II, Reverse Transcription, Promega).

The reaction of real-time PCR was performed in a thermocycler in One Step Real Time PCR system (Applied Biosystems, USA). The starters and conditions used were according to the described by Brietzke et al., 2015 (1). The presence and increase of transcribed for the locus beta of the recipient of T cells (TRB-1), the major histocompatibility complex I and II (MHC-I, MHC-II), the cluster of differentiation 8^{a} (CD8-a), the factor of tumour necrosis alpha (TNF-α), Interferon gamma (IFN-γ) and the elongation factor 1 alpha (ELF 1-α) as reference gene were evaluated. The relative expression of the mRNA was calculated using the method of ΔΔCT adjusting the efficiency of the starters.

TABLE 1
Starters used to evaluate the immune response

| Gen | Forward primer 5′a 3 | Reverse primer 5′ to 3′ | GeneBank no. |
|---|---|---|---|
| ELF 1-a | ACCCTCCTCTTGGTCGTTTC | TGATGACACCAACAGCAACA | AF498320 |
| IFNγ | AAGGGCTGTGATGTGTTTCTG | TGTACTGAGCGGCATTACTCC | NM_001124620 |
| TNFα | GGGGACAAACTGTGGACTGA | GAAGTTCTTGCCCTGCTCTG | AJ277604 |
| CD8α | ACACCAATGACCACAACCATAGAG | GGGTCCACCTTTCCCACTTT | AF178054 |
| MHC II | TGCCATGCTGATGTGCAG | GTCCCTCAGCCAGGTCACT | AF115533 |
| MHC I | TCCCTCCCTCAGTGTCT | GGGTAGAAACCTGTAGCGTG | AY523661 |
| TRB-1 | GTCTTCTGGCAAGTCAACAATGT | GTAAAAGCTGACAATGCAGGTGA | EU072699 |

Tables 2 and 3. Efficacy of the protection of the vaccine against a challenge with P. salmonis by intraperitoneal route

TABLE 2
Relative percent survival at the end of the study (RPS).

| | | | | | Cummulative | | | | |
|---|---|---|---|---|---|---|---|---|---|
| | | | Death | | mortality | Survival | | | |
| Group | Tank | Tank no | numbers | Mortality % | % group | numbers | Survival % | RPS/Tank | RPS mean |
| Control | 1 | 39 | 31 | 79.5 | 83.8 | 8 | 20.5 | | |
| | 2 | 39 | 31 | 79 | | 8 | 20.5 | | |
| | 3 | 40 | 37 | 93 | | 3 | 7.5 | | |
| Vaccine 1 | 1 | 40 | 20 | 50 | 51.7 | 20 | 50.0 | 37.1 | 38.2 |
| | 2 | 40 | 20 | 50 | | 20 | 50.0 | 37.1 | |
| | 3 | 40 | 22 | 55 | | 18 | 45.0 | 40.5 | |
| Vaccine 2 | 1 | 40 | 30 | 75 | 72.5 | 10 | 25.0 | 5.8 | 12.8 |
| | 2 | 40 | 30 | 75 | | 10 | 25.0 | 8.5 | |
| | 3 | 40 | 27 | 68 | | 13 | 32.5 | 27.0 | |

TABLE 3
Relative percent survival when the control group reaches 60% of mortality (RPS60).
RPS 60

| Group | No Total | Mortality No | % Mortality | Survival No | Survival % | Mean RPS |
|---|---|---|---|---|---|---|
| Control | 118 | 74 | 62.8 | 44 | 37.3 | |
| Vaccine 1 | 120 | 25 | 20.8 | 95 | 79.2 | 66.9 |
| Vaccine 2 | 120 | 42 | 35.0 | 78 | 65.0 | 44.3 |

When analysing the efficacy of the treatments, the percent of mortality at final time are the following: Control: 83.8%, Vaccine 1: 51.7%, Vaccine 2: 72.5% (Table 2), achieving significant differences for Vaccine 1 and 2, in relation to the control (Comparison of survival curves: Log-rank). The RPS obtained at final time were the following: Vaccine 1: 38.2%, Vaccine 2: 12.8% (Table 2).

Additionally, the survival percent and mortality analysis at RPS60 per treatment were evaluated. The percent of mortality obtained were the following: Control: 62.8%, Vaccine 1: 20.8%, Vaccine 2: 35% and the estimated RPS60 obtained (average per tank) were: Vaccine 1: 63.7% Vaccine 2: 43.2% (Table 3). When calculating the final RPS60 per treatment the following values are obtained: Vaccine 1: 66.9%, Vaccine 2: 44.3% (Table 4).

The results obtained indicate that the formulations are effective in the protection of vaccinated fish against the salmonid rickettsial syndrome, describing survivals close to 80% (79.2%) in challenges conducted in controlled conditions.

1. Formulation of fish vaccine based on lipidic nanovesicles, especially, a proteoliposome or cochleate, with activity against the Salmonid Rickettsial Syndrome (SRS) CHARACTERIZED THAT it comprises proteoliposomes, membranes and cochleates in a weight ratio of 1:1:1, physiological saline solution and an adjuvant selected from the group of Montanide 760 VG, Montanide 763 AVG, Montanide ISA711, Drakeol 6VR.

2. The formulation of vaccine of claim 1 CHARACTERIZED THAT the quantity of proteoliposome, membrane and cochleates is 20 μg expressed as total protein.

3. The formulation of vaccine of claim 1 CHARACTERIZED THAT the quantity of adjuvant is 60-70 μL.

4. The formulation of vaccine of claim 1 CHARACTERIZED THAT the quantity of sterile physiological saline solution is the sufficient quantity to reach 100 μL of total formulation.

5. Method to prepare a formulation of fish vaccine based on lipidic nanovesicles, especially, a proteoliposome and cochleates, with activity against the Salmonid Rickettsial Syndrome (SRS) CHARACTERIZED THAT it includes:
a) to culture P. salmonis, harvest it by centrifugation and store it frozen;
b) purify membranes and fragments of cell wall from the frozen sediment of the stage a) by resuspending the frozen sediment in a buffer solution adding pearls of zirconia/silica, to then freeze, thaw by sonication and freeze until complete 7 repetitions of the cycle freeze-thaw-freeze, then to centrifugate reserving supernatant and discarding sediment, and then centrifugate reserving sediment and discarding the supernatant,
c) prepare membrane proteoliposomes from the frozen sediment of step b) resuspend the sediment in a membranes solubilisation solution, incubate with agitation, centrifugate and preserve the supernatant, then add non-polar resins able to capture detergent previously resuspended in solution and sterilised, to subsequently incubate with agitation and to add those non-polar resins again to incubate with agitation, leave to settle, preserving the supernatant,
d) prepare membranes from the frozen sediment of step b) resuspend the sediment in a physiological solution or physiological saline, incubate with agitation, centrifugate preserving the supernatant,
e) prepare membrane cochleates from the frozen sediment of stage b) resuspend the sediment in a solubilisation solution (TLis), incubate with agitation, centrifugate and preserve the supernatant. This supernatant is added by dropping at equal volume of formation solution, then 4 volumes of washing solution are added. Finally, the cochleates are centrifuged, discarding the supernatant and the sediment resuspended in washing solution, and
f) the membranes, fragments, membranes proteoliposomes and membrane cochleates obtained in steps c) to e) are mixed with physiological saline, under agitation to an adjuvant and homogenise the mixture, to then optionally store in sealed containers.

6. The method of claim 1 CHARACTERIZED THAT in the step b) said buffer solution is a sterile lysis buffer solution.

7. The method of claim 6 CHARACTERIZED THAT said sterile lysis buffer solution includes disodium phosphate, sodium chloride.

8. The method of claim 1 CHARACTERIZED THAT the ratio of buffer solution (mL) to weight of pearls (g) in the resuspension of stage b) is 25:2 per each gram of sediment obtained.

9. The method of claim 1 CHARACTERIZED THAT in the step c) the membrane solubilization solution includes Tris-HCl, KCl and sodium deoxycholate.

10. The method of claim 1 CHARACTERIZED THAT in the step c), each incubation is conducted at 18-25° C.

11. The method of claim 1 CHARACTERIZED THAT the ratio of supernatant volume (mL) to non-polar resins volume (mL) in the first resuspension of the step c) is 10:1.

12. The method of claim 1 CHARACTERIZED IN THAT the ratio of supernatant volume (mL) to non-polar resins volume (mL) at the end of the step c) is 10:5.

13. The method of claim 1 CHARACTERIZED THAT the ratio of physiological solution or physiological saline (mL) to weight of membrane (g) in step d) is 10:1.

14. The method of claim 1 CHARACTERIZED THAT in the stage f), the ratio of membrane proteoliposomes, membrane and membrane cochleates to physiological saline is 20 μg of total protein to 30-40 μL of sterile physiological saline.

15. The method of claim 1 CHARACTERIZED THAT in the step f), the ratio of volume of the mixture of membrane proteoliposome, membrane, membrane cochleates and physiological saline to adjuvants is 30:70 (it could be 40:60).

16. The method of claim 1 CHARACTERIZED THAT the steps from b) to e) further comprises to verify sterility and concentration of total proteins.

17. The method of claim 1 CHARACTERIZED THAT in the step f) the adjuvant is Seppic 760 VG.

18. The method of claim 1 CHARACTERIZED THAT in the step e) the ratio of the mixture proteoliposome, membrane, cochleate and saline to the adjuvant is 3:7 or 4:6.

19. The method of claim 1 CHARACTERIZED THAT the step f) further comprises to store the formulation of vaccine.
